(19) United States
(12) Patent Application Publication
(10) Pub. No.: US 2007/0010748 A1
(43) Pub. Date: Jan. 11, 2007

(54) Ambulatory monitors

(76) Inventors: Steven D. Rauch, Watertown, MA (US); Conrad Wall III, Boston, MA (US); E. Stuart Tuthill, Newbury, MA (US); Troy Roberts, Pepperell, MA (US)

Correspondence Address: FISH & RICHARDSON PC, P.O. BOX 1022, MINNEAPOLIS, MN, 55440-1022, US

(21) Appl. No.: 11/175,694

(22) Filed: Jul. 6, 2005

(51) Int. Cl. A61B 5/02

(52) U.S. Cl. 600/481

(57) ABSTRACT

An apparatus for recording physiological data from an ambulatory subject. The apparatus includes a sensor circuit configured to detect input analog signals including input analog signals resulting from eye movement of the ambulatory subject, and a signal processing circuit, in communication with the sensor circuit, configured to receive the input analog signals and to generate an output digital signal based at least in part on the received input analog signals. The apparatus has physical characteristics suitable for portable use by the ambulatory subject.

### BACKGROUND

This description relates to ambulatory monitors.

Balance disorders are a prevalent medical problem accounting for billions of healthcare dollars in expenditure each year. The diagnosis of balance disorders often involves monitoring and recording a patient's eye movements, both reflexive and evoked by vestibulo-ocular reflexes, over an extended period of time. Such intensive vestibular monitoring tests are typically conducted in a vestibular laboratory set in a clinical environment by trained technicians who utilize expensive monitoring equipment having multiple sensors that are tethered to a centralized recording system and power supply.

### SUMMARY

In one aspect, the invention features an apparatus for recording physiological data from an ambulatory subject. The apparatus includes a sensor circuit being configured to detect input analog signals including input analog signals resulting from eye movement of the ambulatory subject; and a signal processing circuit in communication with the sensor circuit, the signal processing circuit being configured to receive the input analog signals and to generate an output digital signal based at least in part on the received input analog signals. The apparatus has physical characteristics suitable for portable use by the ambulatory subject.

Implementations of the invention include one or more of the following features. The apparatus includes at least two electrodes coupled to the sensor circuit, the electrodes being adapted for coupling externally to a facial region of the ambulatory subject and detecting an electrooculogram signal. The apparatus includes at least two electrodes coupled to the sensor circuit, the electrodes being adapted for coupling externally to a body region of the ambulatory subject and detecting an electrocardiogram signal resulting from cardiac activity of the ambulatory subject. The apparatus includes a video-based tracking component and/or an infrared reflection component, each being adapted for detecting eye movement of the ambulatory subject.

The apparatus includes a self-contained power source to provide operating power to the apparatus.

The apparatus includes a microprocessor configured to start driving the sensor circuit in response to a first triggering event, to receive the output digital signal from the signal processing circuit, and to store the output digital signal in a memory.

The apparatus includes a switch in communication with the microprocessor. The microprocessor is configured to consider actuation of the switch to be the first triggering event. The microprocessor is also configured to consider a second actuation of the switch to be a second triggering event and to stop driving the sensor circuit in response to the second triggering event.

The microprocessor is configured to stop driving the sensor circuit after a predetermined period of time has elapsed.

The apparatus includes a microprocessor configured to receive the output digital signal from the signal processing circuit, and to store the output digital signal in a first memory. Upon detection of an event trigger, the microprocessor is configured to send the output digital signal from the first memory to a second memory. The first memory can be a temporary storage device and the second memory can be a permanent storage device.

The apparatus includes a receiver in communication with the microprocessor, the receiver being configured to receive a message from a remote device. The microprocessor is configured to consider receipt of the message to be the first triggering event. The microprocessor is also configured to consider receipt of a second message from the remote device to be a second triggering event and to stop driving the sensor circuit in response to the second triggering event.

The apparatus includes a microphone to detect audio, and a microprocessor configured to cause the audio to be stored in a memory and to generate correlation information to temporally correlate the recorded audio with the output digital signal.

The apparatus includes a speaker to output audio, and a microprocessor configured to cause the speaker to playback an instruction.

The apparatus includes a transmitter configured to transmit the output digital signal to a receiver unit for subsequent processing. The transmitter is configured to transmit the output digital signal to the receiver unit at one of a predetermined time, a predetermined condition, or upon demand.

In another aspect, the invention features a system including an ambulatory monitor for use by an ambulatory subject, the ambulatory monitor being configured to generate physiological data relating to eye movement of the ambulatory subject; and a base station unit in communication with the ambulatory monitor, the base station unit being configured to receive physiological data from the ambulatory monitor and to store the physiological data.

Implementations of the invention include one or more of the following features.

The ambulatory monitor includes at least two electrodes being adapted for coupling externally to a facial region of the ambulatory subject and detecting an electrooculogram signal.

The base station unit is configured to receive physiological data from the ambulatory monitor at one of a predetermined time, a predetermined condition, or upon demand. The base station unit is in communication with the ambulatory monitor via a wired and/or wireless communication link.

The system includes a user device in communication with the ambulatory monitor, the user device being configured to remotely control operation of the ambulatory monitor.

In another aspect, the invention features a method including enabling monitoring of an ambulatory subject during activity not subject to limitations associated with the monitoring. The monitoring includes detecting physiological parameters of the ambulatory subject, the physiological parameters including eye movement, and generating physiological data based on the detecting.

Implementations of the invention include one or more of the following features. The monitoring further includes storing the physiological data. The method further includes processing the physiological data in order to aid in a diagnosis of a physiological state of the ambulatory subject (e.g., a balance disorder diagnosis). The physiological parameters can include one or more of a heart rate, pulse rate, beat-to-beat heart variability, electrocardiogram (ECG), respiration rate, skin temperature, and electrooculogram (EOG). The physiological parameters can be associated with movement, position, or both of a body part of the ambulatory subject. The method includes enabling monitoring of the ambulatory subject during activity that is responsive to one or more directions provided to the ambulatory subject.

The invention can be implemented to realize one or more of the following advantages. The ambulatory monitor enables the recording of voluntary and reflexive eye movements and other physiological parameters (e.g., heart rate, respiratory rate, etc.) when the patient is symptomatic and outside of a vestibular laboratory (e.g., during a physical therapy, rehabilitation, or sports medicine context). The recording of physiological parameters other than those directly relating to the patient's eye movements provides a healthcare provider with important insights into the diagnosis of a balance disorder. The ambulatory monitor enables the recording of physiological data that are typically not captured or recorded with current laboratory-based techniques.

The monitor includes a microphone through which the patient provides a synchronized audio recording that sets the context of the physiological data recording. The verbal descriptions correlated with the physiological data can be used by a healthcare provider or another individual to aid in diagnosing the patient's overall physical condition. The monitor includes a speaker (or headset jack for receiving a headset plug) through which auditory instructions can be provided to aid the patient in obtaining relief from the discomfort of the physiological conditions associated with a medical event, e.g., a vestibular symptom attack. The monitor includes two-way communication interfaces which enable a third party, e.g., a healthcare provider, to remotely monitor and assess the patient's physiological state.

The ambulatory monitor is small, lightweight, portable, and is operable by a patient with minimal training. Routine vestibular function testing and monitoring of a patient can be performed by healthcare providers who do not have access to a vestibular laboratory, e.g., in a rural part of the country or in an undeveloped part of the world. Long-term serial monitoring of a patient who is unable or unwilling to make repeated trips to a vestibular laboratory can be performed through the use of the ambulatory monitor. Such long-term serial monitoring enables a healthcare provider to monitor changes to a patient's physical condition over time.

The details of one or more embodiments of the invention are set forth in the accompanying drawings and the description below. Other features and advantages of the invention will become apparent from the description, the drawings, and the claims.

### BRIEF DESCRIPTION OF THE DRAWINGS

FIG. 1 shows a patient monitoring system including an ambulatory monitor.

FIG. 2 shows one implementation of an ambulatory monitor.

### DETAILED DESCRIPTION

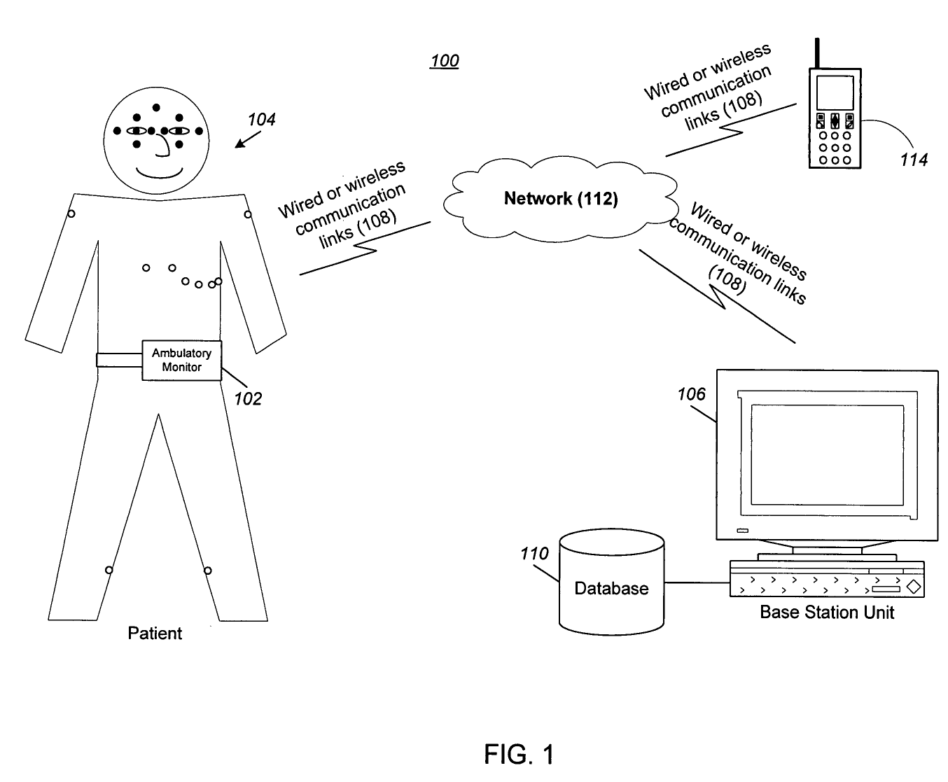

As shown in FIG. 1, a patient monitoring system 100 includes an ambulatory monitor 102 adapted to be placed in proximity to an individual, for example, worn by an ambulatory patient 104 on his body. The monitor 102 collects data (“physiological data”) relating to the patient's physiologic parameters, stores the physiological data in a memory, and optionally, transmits the stored physiological data to a base station unit 106 through communication links 108 of a network 112. Examples of the network 112 include a local area network (“LAN”) and a wide area network (“WAN”), e.g., the Internet, and include both wired and wireless networks. The base station unit 106 may optionally store the physiological data in a database 110 for subsequent processing.

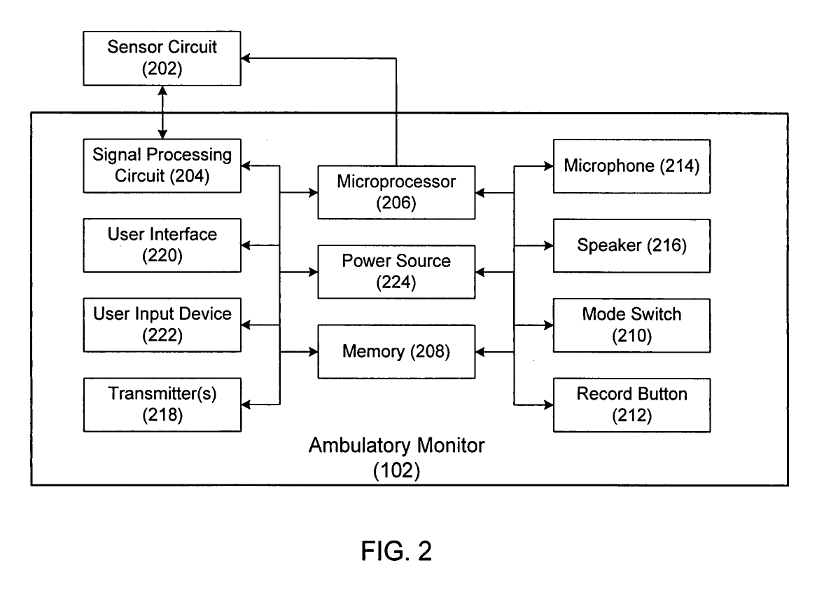

FIG. 2 shows one implementation of the ambulatory monitor 102. The monitor 102 includes a sensor circuit 202 to detect signals indicative of various physiological parameters of the patient 104, such as the patient's heart rate, pulse rate, beat-to-beat heart variability, electrocardiogram (ECG), respiration rate, skin temperature, and electrooculogram (EOG). In the illustrated example, the sensor circuit 202 includes one array of electrodes (represented by filled circles in FIG. 1) that are placed around the eyes of the patient 104, e.g., at the nasal and temporal canthal regions, as well as above and below each eye to detect the EOG signals resulting from the patient's voluntary and reflexive eye movements. The EOG signals record a corneal-retinal potential (voltage difference between the metabolically active retina and relatively quiescent cornea). The EOG signals are a result of a number of factors, including eyeball rotation and movement, eyelid movement, and different sources of artifact such as electrode placement, head movements, influence of ambient illumination, etc. Such artifacts can be reduced or eliminated in further processing steps (as described below with reference to a filtering component of the signal processing circuit 204) so as to produce EOG signals that accurately represent the patient's eye movements. In other implementations, the ambulatory monitor 102 records eye movements of the patient 104 using techniques other than by detection of EOG signals, for example, infrared reflection, and video tracking.

In the illustrated example, the sensor circuit 202 also includes another array of electrodes (represented by unfilled circles in FIG. 1) that are placed on the patient's chest and limbs in order to detect the ECG signals resulting from the patient's cardiac activity. The sensor circuit 202 samples the EOG signals and the ECG signals at a 200 Hz sampling frequency with a data resolution of 12 or 16 bits. Methods for detecting signals indicative of various physiological parameters of a patient and electrodes that are used in detecting are well known. Examples of such signals include electromyography (EMG) signals, electroencephalography (EEG) signals, arterial blood oxygen saturation (SaO₂) signals, and respiratory signals. In some implementations, the sensor circuit 202 further detects the position and/or movement of various body parts of the patient 104, including but not limited to the patient's head, legs, torso, and arms.

The detected signals, including but not limited to the EOG and ECG signals, are provided as analog input signals to a signal processing circuit 204 over electrode lead wires (not shown). The signal processing circuit 204 includes an array of amplifiers that amplify the analog input signals. In one implementation, the amplifiers are two-channel, input buffered, differential-input instrumentation amplifiers sampling at a rate of 200 Hz with high input impedance (e.g., 1 MΩ), high common-mode rejection ratio (e.g., greater than 110 dB), low noise (e.g., less than 2 μV), and appropriate isolation to safeguard the user from electrical shock. The amplifier outputs are filtered (e.g., using lowpass filters with a frequency bandwidth 0 Hz-40 Hz), interleaved, and subsequently converted into a digital signal by other components (e.g., an array of lowpass filters, a multiplexer, and an analog-to-digital converter) of the signal processing circuit 204.

The digital signal is then sent to a microprocessor 206 (e.g., a Hitachi SH-3 RISC microprocessor 206 available from Hitachi Ltd). The microprocessor 206 is programmed to use the digital signal to derive data indicative of at least one aspect of the patient's physiological state, and to subsequently format that derived physiological data into a predetermined format (e.g., European Data Format (EDF)) for data storage in a memory 208 (e.g., a flash memory 208). Thus, over time, the memory 208 of the monitor 102 accumulates a store of physiological data.

The monitor 102 can be implemented with a mode switch 210 that switches between “normal” mode and “standby” mode. When the monitor 102 operates in a “normal” mode, it collects physiological data continuously. Such a recording is referred to in this description as a “continuous recording.” Continuous recordings provide data indicative of one or more aspects of the patient's physiological state before, during, and after the patient experiences symptoms consistent with a balance-disorder episode. Such a recording provides a third party (e.g., a healthcare provider with important insights into the diagnosis of a balance disorder).

Continuous recordings are often used in a clinical environment in which the patient's activities are supervised or dictated in an attempt to evoke certain physiological responses. For example, a physical therapist may require the patient 104 to use the ambulatory monitor 102 to collect physiological data that may subsequently be used to determine the patient's rehabilitation progress. In another example, a coach or physical trainer may require an athlete to use the ambulatory monitor 102 to collect exercise-induced physiological data that may subsequently be used to provide training and/or performance feedback.

When the monitor 102 operates in “standby” mode, it waits for a first event trigger. Upon detection of a first event trigger, the microprocessor 206 drives the sensor circuit 202 to transition the monitor 102 from the “standby” mode to the “normal” mode. One example of a first event trigger is the detection of a record button 212 being pressed. The monitor 102 collects data until the occurrence of a second event trigger. Examples of a second event trigger include the detection of the record button 212 being pressed for a second time to signal a return to the “standby” mode, or the lapse of a predetermined recording period. Such a recording is referred to in this description as an “event-based recording” and is generally used in an ambulatory environment in which the patient 104 is attending to his regular day-to-day activities and experiences symptoms consistent with a balance disorder, abnormal cardiac activity, and the like.

In another implementation, the microprocessor 206 includes one or more algorithms for detecting an event trigger that is caused by an external force other than the patient 104 depressing the record button 212, and driving the sensor circuit 202 to transition the monitor 102 from the “standby” mode to the “normal” mode.

In some implementations, the monitor 102 includes a temporary memory store in addition to the memory 208. When the monitor 102 operates in “standby” mode, the microprocessor 102 continuously writes the derived physiological data to the temporary memory store, which in one example, is configured to store the most recent ten minutes of derived physiological data. Upon detection of a first event trigger, the microprocessor 206 drives the sensor circuit 202 to transition the monitor 102 from the “standby” mode to the “normal” mode, transfers the data stored in the temporary store to the memory 208, and continuously writes the derived physiological data to the memory 208 for storage until the occurrence of a second event trigger. The physiological data collected prior to the detection of the first event trigger may subsequently be used to provide a more complete report of the context of the patient's physiological state. The monitor 102 also includes a microphone 214 and a speaker 216 that can be used respectively to record and play audio at selected times (e.g., in response to indications received from the patient 104 and/or the microprocessor 206 as described below). The microphone 214 can be integral with the monitor 102, or located outside a housing of the monitor 102 and worn, by the patient (e.g., on a wrist, arm or waist of the patient 104). The microprocessor 206 can be implemented to cause audio detected by the microphone 214 during a continuous or event-based recording and to be stored in the memory 208.

If audio is detected, the microprocessor 206 generates correlation information (e.g., time stamps) to temporally correlate the detected audio with the physiological data. Storing audio with the physiological data allows for a more accurate and complete report of the context of the patient's physiological state to be generated and subsequently used by a third party (e.g., a healthcare provider or a physical trainer) in diagnosing the patient's physical condition. The storage of audio allows the patient 104 to provide verbal descriptions of the circumstances he is currently experiencing. Such circumstances may include his surroundings and location, or how he feels at that moment. The patient can provide such a description in a free-flow format or in response to pre-stored cues that are played over the speaker 216. The microprocessor 206 can be programmed to select the cues and sequence of cues to be played based in part on the type, trend, or inferences that can be made on the basis of the collected physiological data. The microprocessor 206 can also be programmed to play audio instructions over the speaker 216 in response to the detection of certain threshold violations. For example, if the physiological data related to a detected signal falls below a threshold value for a period of time, the microprocessor 206 signals this occurrence as an event and automatically causes a sequence of pre-stored instructions to be played over the speaker 216 to aid the patient 104 in obtaining relief from the discomfort of the physiological conditions associated with the event.

The data stored in memory 208, or selected portions thereof, is periodically uploaded from the monitor 102 and sent to the base station unit 106, where it is stored in the database 110 for subsequent processing (e.g., by EOG analyzer software at the base station unit 106) and presentation to the patient 104 or a third party. This uploading of data can be an automatic process that is initiated by the monitor 102 periodically (e.g., every 24 hours for continuous recordings), after each event-based recording, or it can be a manual process initiated by the patient 104 or a third party. The monitor 102 and the base station unit 106 may exchange data over wireless communication links 108 (e.g., via a radio frequency communication or an infrared communication) or wired communication links 108 (e.g., USB IEEE 1394 interfaces) depending on the transmitter interfaces 218 implemented on the monitor 102.

Alternatively, rather than storing the physiological data and recorded data in the memory 208, the monitor 102 continuously uploads the data to the base station unit 106 in real time.

In addition to uploading data to the base station unit 106, the network 112 may be used to facilitate two-way communication between the monitor 102, the base station unit 106, and a user device 114 (illustratively shown as a mobile telephone) associated with a third party, e.g., a healthcare provider. The user device can be a pager, a personal digital assistants (“PDA”), a portable personal communicator (such as a mobile communicator), a laptop, a desktop, or any of a variety of other two-way communication devices. In one example, the healthcare provider remotely monitors and assesses the patient's physiological state by obtaining (via the user device 114) physiological data and/or recorded audio from the monitor 102 and/or the base station unit 106. In another example, the monitor 102 sends an alert to the user device 114 if physiological data related to a detected signal falls below a threshold value for a period of time, possibly signaling the occurrence of a balance disorder episode. In such a scenario, the healthcare provider can selectively override the playback of the sequence of pre-stored instructions over the speaker 216, and provide verbal and/or text-based instructions (via the user device 114) in real-time to aid the patient 104 in obtaining relief from the discomfort of the physiological conditions associated with the episode. In a third example, the healthcare provider can remotely switch the operation mode of the monitor 102 from a “standby” mode to a “normal mode” and vice versa by pressing a button on the user device 114 or otherwise providing a user input that results in the switch in operation mode.

The physiological data stored in the memory 208 can be presented to the patient 104 or a third party via a user interface 220 of the monitor 102. For this purpose, the monitor 102 can include a display, such as a light emitting diode (LED) display or a liquid crystal display (LCD). The patient 104 or the third party can selectively retrieve and display physiological data associated with different aspects of the patient's physiological state via a user input device 222 of the monitor 102, such as a keypad or a pointing device. If audio was stored along with the physiological data, the patient 104 or the third party can listen to the recorded audio over the speaker 216.

The monitor 102 is powered by a self-contained power source 224, e.g., a rechargeable battery pack or disposable batteries. The microprocessor 206 can be implemented to monitor 102 the battery levels and indicate that the batteries need to be replaced or recharged, e.g., using a series of LEDs located on the monitor 102, a battery charge level meter on the LCD, or by transmitting a low battery message to the base station unit 106.

The monitor 102 has physical characteristics suitable for portable use by the patient 104. The monitor 102 is sized so that it can be comfortably worn on the patient's body (e.g., on a belt around the patient's waist, over a shoulder, or around a thigh) or toted around in close proximity to the patient 104 (e.g., in a bag, or attached to a walker) during the course of the patient's regular day-to-day activity.

The invention has been described in terms of particular embodiments. Other embodiments are within the scope of the following claims. The following are examples for illustration only and not to limit the alternatives in any way. The techniques described herein can be performed in a different order and still achieve desirable results.

What is claimed is:

1. An apparatus for recording physiological data from an ambulatory subject, the apparatus comprising:
a sensor circuit being configured to detect input analog signals including input analog signals resulting from eye movement of the ambulatory subject; and
a signal processing circuit in communication with the sensor circuit, the signal processing circuit being configured to receive the input analog signals and to generate an output digital signal based at least in part on the received input analog signals;
wherein the apparatus has physical characteristics suitable for portable use by the ambulatory subject.

2. The apparatus of claim 1, further comprising:
at least two electrodes coupled to the sensor circuit, the electrodes being adapted for coupling externally to a facial region of the ambulatory subject and detecting an electrooculogram signal.

3. The apparatus of claim 1, wherein the sensor circuit comprises an infrared reflection component.

4. The apparatus of claim 1, wherein the sensor circuit comprises a video-based tracking component.

5. The apparatus of claim 1, further comprising:
at least two electrodes coupled to the sensor circuit, the electrodes being adapted for coupling externally to a body region of the ambulatory subject and detecting an electrocardiogram signal resulting from cardiac activity of the ambulatory subject.

6. The apparatus of claim 1, further comprising:
a self-contained power source to provide operating power to the apparatus.

7. The apparatus of claim 1, further comprising:
a microprocessor configured to start driving the sensor circuit in response to a first triggering event, to receive the output digital signal from the signal processing circuit, and to store the output digital signal in a memory.

8. The apparatus of claim 7, further comprising:
a switch in communication with the microprocessor, and wherein the microprocessor is configured to consider actuation of the switch to be the first triggering event.

9. The apparatus of claim 8, wherein the microprocessor is configured to consider a second actuation of the switch to be a second triggering event and to stop driving the sensor circuit in response to the second triggering event.

10. The apparatus of claim 7, wherein the microprocessor is configured to stop driving the sensor circuit after a predetermined period of time has elapsed.

11. The apparatus of claim 7, further comprising:
a receiver in communication with the microprocessor, the receiver being configured to receive a message from a remote device, and wherein the microprocessor is configured to consider receipt of the message to be the first triggering event.

12. The apparatus of claim 11, wherein the microprocessor is configured to consider receipt of a second message from the remote device to be a second triggering event and to stop driving the sensor circuit in response to the second triggering event.

13. The apparatus of claim 1, further comprising:
a microprocessor configured to receive the output digital signal from the signal processing circuit, and to store the output digital signal in a first memory, whereby upon detection of an event trigger, the microprocessor is configured to send the output digital signal from the first memory to a second memory.

14. The apparatus of claim 13, wherein the first memory is a temporary storage device and the second memory is a permanent storage device.

15. The apparatus of claim 1, further comprising:
a microphone to detect audio; and
a microprocessor configured to cause the audio to be stored in a memory and to generate correlation information to temporally correlate the recorded audio with the output digital signal.

16. The apparatus of claim 1, further comprising:
a speaker to output audio; and
a microprocessor configured to cause the speaker to playback an instruction.

17. The apparatus of claim 1, further comprising:
a transmitter configured to transmit the output digital signal to a receiver unit for subsequent processing.

18. The apparatus of claim 17, wherein the transmitter is configured to transmit the output digital signal to the receiver unit at one of a predetermined time, a predetermined condition, or upon demand.

19. A system comprising:
an ambulatory monitor for use by an ambulatory subject, the ambulatory monitor being configured to generate physiological data relating to eye movement of the ambulatory subject; and
a base station unit in communication with the ambulatory monitor, the base station unit being configured to receive physiological data from the ambulatory monitor and to store the physiological data.

20. The system of claim 19, wherein the ambulatory monitor comprises at least two electrodes being adapted for coupling externally to a facial region of the ambulatory subject and detecting an electrooculogram signal.

21. The system of claim 19, wherein the ambulatory monitor comprises an infrared reflection component being adapted for detecting eye movement of the ambulatory subject.

22. The system of claim 19, wherein the ambulatory monitor comprises a video-based tracking component being adapted for detecting eye movement of the ambulatory subject.

23. The system of claim 19, wherein the base station unit is configured to receive physiological data from the ambulatory monitor at one of a predetermined time, a predetermined condition, or upon demand.

24. The system of claim 19, further comprising:
a user device in communication with the ambulatory monitor, the user device being configured to remotely control operation of the ambulatory monitor.

25. The system of claim 19, wherein the base station unit is in communication with the ambulatory monitor via a wired communication link.

26. The system of claim 19, wherein the base station unit is in communication with the ambulatory monitor via a wireless communication link.

27. A method comprising:
enabling monitoring of an ambulatory subject during activity not subject to limitations associated with the monitoring, the monitoring comprising detecting physiological parameters of the ambulatory subject, the physiological parameters including eye movement, and generating physiological data based on the detecting.

28. The method of claim 27, wherein the monitoring further comprises storing the physiological data.

29. The method of claim 27, further comprising:
processing the physiological data in order to aid in a diagnosis of a physiological state of the ambulatory subject.

30. The method of claim 29, wherein the diagnosis comprises a balance disorder diagnosis.

31. The method of claim 27, wherein the physiological parameters comprise one or more of a heart rate, pulse rate, beat-to-beat heart variability, electrocardiogram (ECG), respiration rate, skin temperature, and electrooculogram (EOG).

32. The method of claim 27, wherein the physiological parameters are associated with movement, position, or both of a body part of the ambulatory subject.

33. The method of claim 27, wherein enabling monitoring comprises enabling monitoring of the ambulatory subject during activity that is responsive to one or more directions provided to the ambulatory subject.
